(19) United States
(12) Patent Application Publication
(10) Pub. No.: US 2006/0052870 A1
(43) Pub. Date: Mar. 9, 2006

(54) Methods and apparatus to prevent movement through artificial disc replacements

(76) Inventor: Bret A. Ferree, Cincinnati, OH (US)

Correspondence Address: John G. Posa;Gifford, Krass, Groh, Sprinkle,, Anderson & Citkowski, P.C., PO Box 7021, Troy, MI, 48007-7021, US

(21) Appl. No.: 11/099,090

(22) Filed: Apr. 5, 2005

Related U.S. Application Data
(60) Provisional application No. 60/608,401, filed on Sep. 9, 2004.

(51) Int. Cl. A61F 2/44; A61B 17/70

(52) U.S. Cl. 623/17.11; 606/61

(57) ABSTRACT

This invention is directed to improved methods and devices to attach arthroplasty devices, particularly the spine, distract the disc space, machine the disc space to improve the fit between ADRs and the vertebrae, to hold and remove ADRs, and to facilitate spinal fusion for a failed ADR surgery. One aspect of the invention places anchor devices in the spine during a first operation. Spinal devices are connected to the anchoring devices during a second procedure. The second procedure is generally performed months after the insertion of the anchoring devices. The time between the two procedures allows bone to grow into the anchoring devices. Minimal forces are exerted on the anchoring devices between the procedures. Other embodiments are described where a previously implanted ADR having a pair of opposing endplates is immobilized using a device the attached to the endplates, fits between the endplates, or both.

### REFERENCE TO RELATED APPLICATIONS

This application claims priority from U.S. Provisional Patent Application Ser. No. 60/608,401, filed Sep. 9, 2004, the entire content of which is incorporated herein by reference.

### FIELD OF THE INVENTION

This invention relates generally to surgical methods and apparatus and in particular to methods and apparatus for preventing movement through artificial disc replacements.

### BACKGROUND OF THE INVENTION

A small percent of patients will continue to experience low back pain (LBP) following insertion of Artificial Disc Replacements (ADR). Surgeons often fuse the vertebrae immediately above and below the ADR in an attempt to decrease patients' LBP. Patients' spine may be fused from a posterior approach to the spine. Pedicle screws and other types of spinal instrumentation may be used to facilitate fusion. Many surgeons believe a posterior spinal fusion is inadequate following ADR insertion. They believe continued movement through the ADR will prevent a successful fusion. Thus many surgeons advocate removal of the ADR and spinal fusion with interbody cages and pedicle screws. All surgeons agree that removal of ADRs, particularly in the lumbar spine, is dangerous procedure. The great vessels including the vena cava, aorta, and iliac vessels may become adherent to the spine following insertion of ADRs. The fragile vessels may be torn during revision surgery. Tears of the great vessels may be life threatening.

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction). The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

Interestingly, the adult disc is the largest avascular structure in the human body. Given the lack of vascularity, the nucleus is not exposed to the body's immune system. Most cells in the nucleus obtain their nutrition and fluid exchange through diffusion from small blood vessels in adjacent vertebra.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised.

The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration include procedures to remove the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. Spinal fusion procedures either remove the disc or the disc's function by connecting two or more vertebra together with bone. Perhaps the most promising solution, prosthetic disc replacement, offers many advantages. The prosthetic disc attempts to eliminate a patient's pain while preserving the disc's function.

Prior-art techniques connect stabilization and arthroplasty devices to the anchoring components during a single procedure. The prior-art technique subjects the anchoring devices to excessive forces. Excessive forces on the anchoring devices frequently cause movement between the vertebrae and the anchoring components. Movement between the anchoring component and the vertebrae inhibits bone ingrowth. Thus, despite advances in this field, the need remains for further improvements in the way in which prosthetic components are incorporated into the disc space, and in materials to ensure strength and longevity.

### SUMMARY OF THE INVENTION

This invention is directed to improved methods and devices to attach arthroplasty devices, particularly the spine, distract the disc space, machine the disc space to improve the fit between ADRs and the vertebrae, to hold and remove ADRs, and to facilitate spinal fusion for a failed ADR surgery.

One aspect of the invention places anchor devices in the spine during a first operation. Spinal devices are connected to the anchoring devices during a second procedure. The second procedure is generally performed months after the insertion of the anchoring devices. The time between the two procedures allows bone to grow into the anchoring devices. Minimal forces are exerted on the anchoring devices between the procedures.

The anchor devices used in the novel invention are manufactured to promote bone ingrowth in the preferred embodiment of the device. For example, the anchoring devices may be covered with titanium plasma spray or hydroxyapatite. Alternatively, the anchoring devices could be made of tantalum (Zimmer). The anchoring devices are preferably placed with a minimally invasive surgical (MIS) procedure.

Other embodiments are described where a previously implanted ADR having a pair of opposing endplates is immobilized using a device the attached to the endplates, fits between the endplates, or both.

### BRIEF DESCRIPTION OF THE DRAWINGS

FIG. 1A shows a sagittal cross section of the spine and screws used to anchor a spinal arthroplasty device;

FIG. 1B shows a sagittal cross-section of the spine and a dynamic stabilization device;

FIG. 2 shows a sagittal cross-section of the spine, anchoring components, and a facet replacement device;

FIG. 3A shows a coronal cross-section of the spine and an artificial disc replacement (ADR);

FIG. 3B shows a sagittal cross-section of the spine and the embodiment of the invention shown in FIG. 3A;

FIG. 3C shows an anterior view of the spine and an exploded view of the ADR and staple shown in FIG. 3A;

FIG. 3D shows an inferior view of the inferior ADR component;

FIG. 3E shows a sagittal cross-section of the ADR and staple shown in FIG. 3B;

FIG. 3F shows a coronal cross-section of the spine and an alternative embodiment of the invention;

FIG. 4A is an anterior view of the spine, an exploded view of an alternative embodiment of an ADR incorporating novel spikes;

FIG. 4B shows a sagittal cross-section of the spine and the embodiment of the invention shown in FIG. 4A;

FIG. 4C shows a coronal cross-section of the spine and the embodiment of the invention shown in FIG. 4A;

FIG. 4D shows a coronal cross-section of the spine and an alternative embodiment of the invention shown in FIG. 4C;

FIG. 5A shows an anterior view of a novel cutting and distraction device;

FIG. 5B shows a lateral view of the embodiment of the device shown in FIG. 5A;

FIG. 5C shows a lateral view of the device shown in FIG. 5B;

FIG. 5D shows an exploded lateral view of the embodiment of the invention shown in FIG. 5C;

FIG. 5E shows a view of the top of the cutting guide shown in FIG. 5A;

FIG. 5F shows a view of the top of an alternative cutting guide;

FIG. 5G shows a sagittal cross-section of the spine and a portion of the device shown in FIG. 5C;

FIG. 5H shows a sagittal cross-section of the spine, a portion of the device shown in FIG. 5G;

FIG. 5I shows a sagittal cross-section of the spine, a portion of the device shown in FIG. 5G, a prior-art distraction device, a portion of a saw and saw blade;

FIG. 5J shows a superior view of the cutting guide, a portion of the holding tool, and the saw shown in FIG. 5I;

FIG. 6A shows a lateral view of the spine and a novel guide;

FIG. 6B shows a partial sagittal view of the spine and the novel guide shown in FIG. 6A;

FIG. 6C shows a partial sagittal view of the spine after insertion of the distraction screws;

FIG. 6D shows a partial sagittal view of the spine, distraction screws, and a novel distraction apparatus;

FIG. 6E shows partial sagittal view of the spine, the embodiment of the invention drawn in FIG. 6D, an ADR, and a novel ADR holder;

FIG. 7A shows superior view of an ADR and an alternative embodiment of the present invention;

FIG. 7B shows a superior view of a novel “slap hammer” that may be used with the holder shown in FIG. 7A to extract the ADR;

FIG. 7C shows a view of the assembled devices shown in FIGS. 7A and 7B;

FIG. 7D shows an exploded lateral view of an ADR and a portion of the pliers-like device shown in FIG. 7A;

FIG. 7E shows a lateral view of an assembled ADR and holder;

FIG. 7F shows a lateral view of the spine and the ADR and holder shown in FIG. 7E;

FIG. 8A shows a lateral view of the cutting guide shown in FIG. 5F;

FIG. 8B shows a coronal cross-section of the cutting guide shown in FIG. 8A;

FIG. 8C shows a lateral view of the spine, and the embodiment of the device drawn in FIG. 8B;

FIG. 9A shows an anterior view of a novel component used to prevent movement of ADRs;

FIG. 9B shows a lateral view of the device shown in FIG. 9A;

FIG. 9C shows a lateral view of an ADR and the device shown in FIG. 9B;

FIG. 9D shows an exploded lateral view of the embodiment of the invention drawn in FIG. 9C;

FIG. 9E shows a partial sagittal view of the spine, the embodiment of the invention drawn in FIG. 9C, and plates and screws;

FIG. 10A shows a lateral view of a novel ADR;

FIG. 10B shows a lateral view of an alternative embodiment;

FIG. 11A shows an anterior view of an alternative embodiment of the ADR shown in FIG. 10B;

FIG. 11B shows a lateral view of the ADR drawn in FIG. 11A;

FIG. 11C shows an anterior view of the spine;

FIG. 12 shows a superior view of a pliers-like holder that fits into triangular or other shaped depressions in the sides of the ADR;

FIG. 13A shows a lateral view of an alternative distractor/cutting guide according to the present invention;

FIG. 13B shows a lateral view of the spine and the device;

FIG. 13C shows a partial sagittal cross-section of the spine and the device shown in FIG. 13B;

FIG. 13D shows a lateral view of the spine;

FIG. 14A shows a lateral view of the spine and an alternative distractor/guide;

FIG. 14B shows a partial sagittal cross-section of the spine and the device shown in FIG. 14A;

FIG. 14C shows a view of the top of a portion of the guide shown in FIG. 14A;

FIG. 14D shows a view of the top of the guide shown in FIG. 14C;

FIG. 15A shows an anterior view of an alternative ADR including screws used to attach the ADR to the vertebra above and below the ADR;

FIG. 15B shows a lateral view of the ADR shown in FIG. 15B;

FIG. 16A shows an anterior view of an alternative embodiment of the present invention;

FIG. 16B shows a lateral view of the spine and the embodiment of the ADR shown in FIG. 16A;

FIG. 16C shows a lateral view of the spine and an alternative embodiment of the present invention;

FIG. 17A shows a view of the bottom of the upper ADR endplate (ADR EP) shown in FIG. 16A;

FIG. 17B shows a view of the top of the lower ADR EP shown in FIG. 16B;

FIG. 17C shows a coronal cross-section of the ADR shown in FIG. 16A;

FIG. 17D shows a lateral view of an instrument used to align the ADR EPs shown in FIG. 17C;

FIG. 17E shows a coronal cross-section of the ADR shown in FIG. 17C and the alignment tool drawn in FIG. 17D;

FIG. 18 shows an anterior view of an alternative embodiment of the present invention;

FIG. 19A shows an exploded lateral view of an ADR and alternative embodiment of the present invention related to FIG. 9D;

FIG. 19B shows a lateral view of the embodiment of the invention shown in FIG. 19A and an ADR;

FIG. 19C shows a posterior view of the device and an ADR;

FIG. 20 shows the view of the top of an ADR and embodiments of the invention shown in FIG. 19A;

FIG. 21 shows a posterior view of an ADR having holes to receive the embodiment of the invention shown in FIG. 19A;

FIG. 22 shows a lateral view of an alternative embodiment of the invention and a sagittal cross-section through a novel ADR;

FIG. 23A shows an exploded lateral view of an alternative embodiment of the present invention;

FIG. 23B shows a lateral view of the embodiment of the invention shown in FIG. 23A and an ADR;

FIG. 24A shows an exploded lateral view of an alternative embodiment of the invention shown in FIG. 23A;

FIG. 24B shows a lateral view of the embodiment of the invention shown in FIG. 24A and a novel ADR;

FIG. 24C shows a view of the top of the embodiment of the ADR shown in FIG. 24B;

FIG. 24D shows a posterior view of the embodiment of the invention shown in FIG. 24C;

FIG. 25A shows an exploded lateral view of an alternative embodiment of the present invention;

FIG. 25B shows a sagittal cross-section of the embodiment of the present invention shown in FIG. 25A and an ADR;

FIG. 26A shows an exploded lateral view of an alternative embodiment of the present invention;

FIG. 26B shows a lateral view of the embodiment of the invention drawn in FIG. 26A and a sagittal cross-section through an ADR;

FIG. 27A shows an exploded lateral view of an alternative embodiment of the present invention and an ADR;

FIG. 27B shows a lateral view of the embodiment of the invention shown in FIG. 27A;

FIG. 27C shows a posterior view of the embodiment of the invention shown in FIG. 27B;

FIG. 27D shows a partial posterior view of an alternative embodiment of the present invention related to that shown in FIG. 27C;

FIG. 28A shows an exploded lateral view of an alternative embodiment of the present invention;

FIG. 28B shows an exploded view of the top of the embodiment of the present invention shown in FIG. 28A;

FIG. 28C shows a lateral view of the embodiment of the invention shown in FIG. 28B and an ADR;

FIG. 28D shows a lateral view of the embodiment of the invention shown in FIG. 28C;

FIG. 28E shows a view of the top of an ADR EP and the embodiment of the present invention shown in FIG. 28B;

FIG. 28F shows a lateral view of the embodiment of the invention shown in FIG. 28E;

FIG. 29A shows a lateral view of an alternative embodiment of the present invention;

FIG. 29B shows a lateral view of the embodiment of the invention shown in FIG. 29A;

FIG. 29C shows a sagittal cross-section of the embodiment of the invention shown in FIG. 29B;

FIG. 30A shows a lateral view of the tip of an alternative embodiment of the present invention;

FIG. 30B shows a lateral view of the embodiment of the invention shown in FIG. 30A;

FIG. 30C shows a lateral view of the embodiment of the invention shown in FIG. 30B and an ADR;

FIG. 31A shows a lateral view of an alternative embodiment of the device shown in FIG. 25A;

FIG. 31B shows a view of the top of the inferior ADR EP shown in FIG. 31A;

FIG. 31C shows a view of the top of the inferior ADR EP shown in FIG. 31B;

FIG. 32A shows a view of the articulating side of the ADR EP shown in FIG. 27A;

FIG. 32B shows a view of the articulating side of an alternative embodiment of the invention shown in FIG. 32A;

FIG. 32C shows a view of the articulating side of an alternative embodiment of the present invention;

FIG. 32D shows a view of the articulating side of an alternative embodiment of the invention shown in FIG. 32C;

FIG. 33 shows a lateral view of an ADR and an alternative embodiment of the present invention;

FIG. 34A shows a lateral view of an alternative embodiment of the present invention;

FIG. 34B shows a lateral view of the embodiment of the invention shown in FIG. 34A;

FIG. 34C shows a lateral view of an ADR and the embodiment of the invention shown in FIG. 34B;

FIG. 35 shows the view of the top of an alternative embodiment of the invention shown in FIG. 27A;

FIG. 36A shows an oblique view of an alternative embodiment of the invention shown in FIG. 19A;

FIG. 36B shows a view of the posterior aspect of an alternative embodiment of the ADR shown in FIG. 5A of co-pending U.S. patent application Ser. No. 10/741,290, the entire content of which is incorporated herein by reference;

FIG. 36C shows a view of the posterior aspect of the embodiment of the ADR shown in FIG. 36B and the posterior aspect of the embodiment of the invention drawn in FIG. 36A; and

FIG. 36D shows a sagittal cross section through the embodiment of the ADR drawn in FIG. 36B.

### DETAILED DESCRIPTION OF THE INVENTION

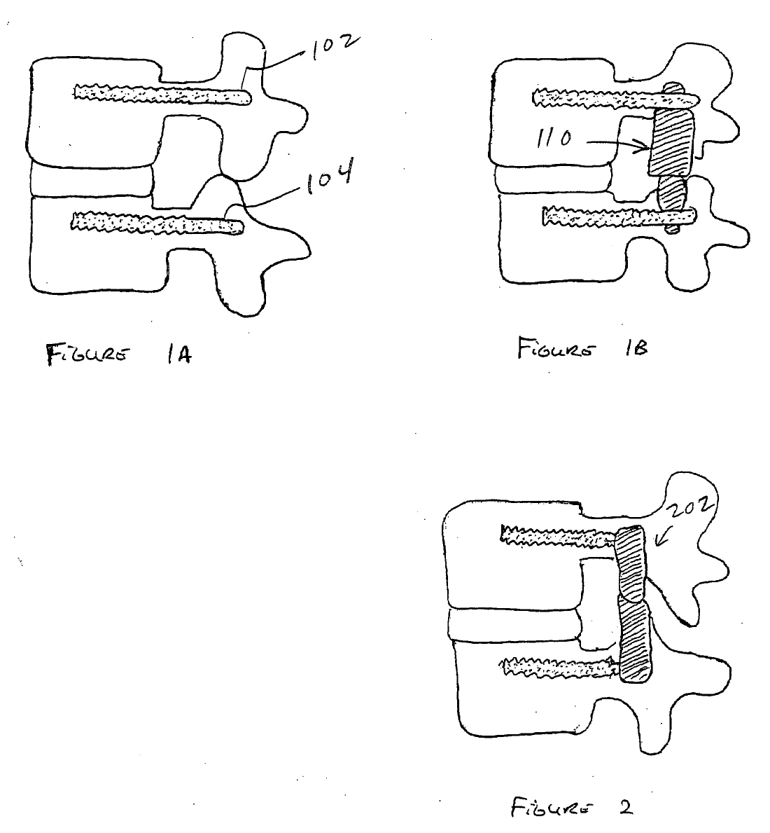

FIG. 1A is a sagittal cross section of the spine and screws 102, 104 used to anchor a spinal arthroplasty device (not shown). The screws are partially contained in the pedicles of the vertebrae. The screws, or other anchoring devices, could be placed in other areas of the vertebrae. FIG. 1B is a sagittal cross section of the spine and a dynamic stabilization device 110. A suitable device is taught in my co-pending U.S. patent application Ser. No. 10/412,896, the entire content of which is incorporated herein by reference. The dynamic stabilization device may be connected to the pedicle screws during a second operation several months after inserting screws 102,104.

FIG. 2 is a sagittal cross section of the spine, anchoring components, and a facet replacement device 202 attached to the anchoring components during a second procedure after inserting the anchoring components.

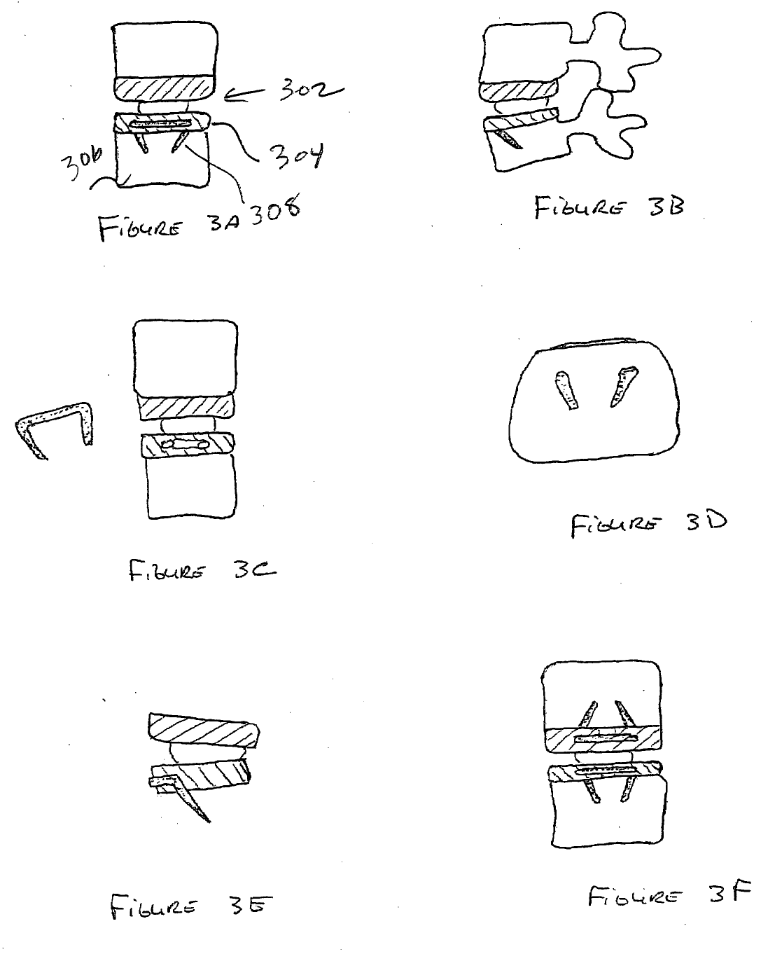

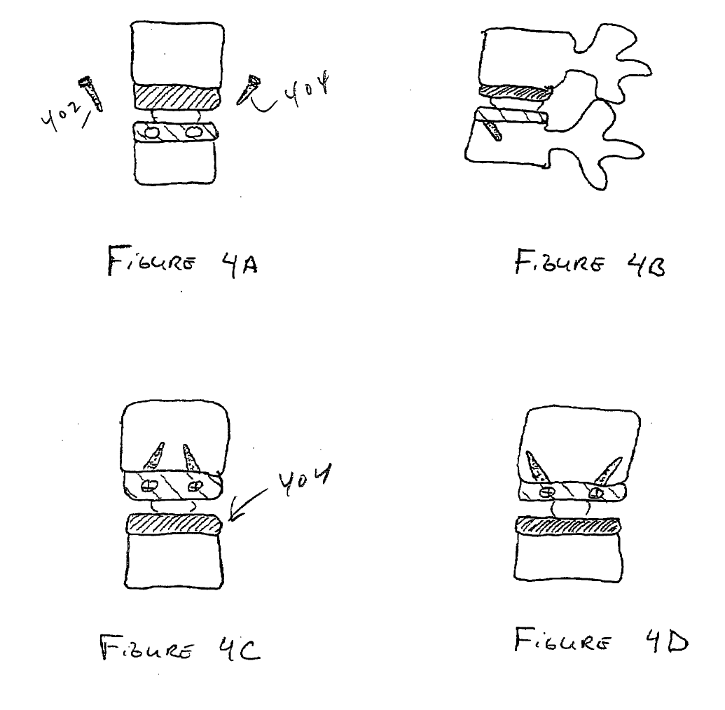

FIG. 3A is a coronal cross section of the spine and an artificial disc replacement (ADR) 302. The inferior ADR component 304 is attached to the inferior vertebra 306 with a novel staple 308. The holes in the ADR force the arms of the staple to converge as the staple is driven into the ADR. Alternatively, the holes in the ADR could force the arms of the staple to diverge as the staple is driven into the ADR. The novel attachment device does not project anterior to the spine. Prior-art cervical ADRs attach the ADRs to the front of the spine. Devices that extend unto the anterior surface of the cervical spine place pressure on the esophagus. The staple may be made of a shape memory material such as Nitinol. FIG. 3B is a sagittal cross section of the spine and the embodiment of the invention drawn in FIG. 3A showing how the arms of the staple course through the endplate of the vertebra below the ADR.

FIG. 3C is an anterior view of the spine and an exploded view of the ADR and staple drawn in FIG. 3A. FIG. 3D is an inferior view of the inferior ADR component. The arms of the staple are seen projecting from holes in the ADR. FIG. 3E is a sagittal cross section of the ADR and staple drawn in FIG. 3B. The holes in the ADR force the arms of the staple to bend inferiorly as the staple is driven into the ADR. FIG. 3F is a coronal cross section of the spine and an alternative embodiment of the invention, wherein both ADR components are attached to the vertebrae with staples.

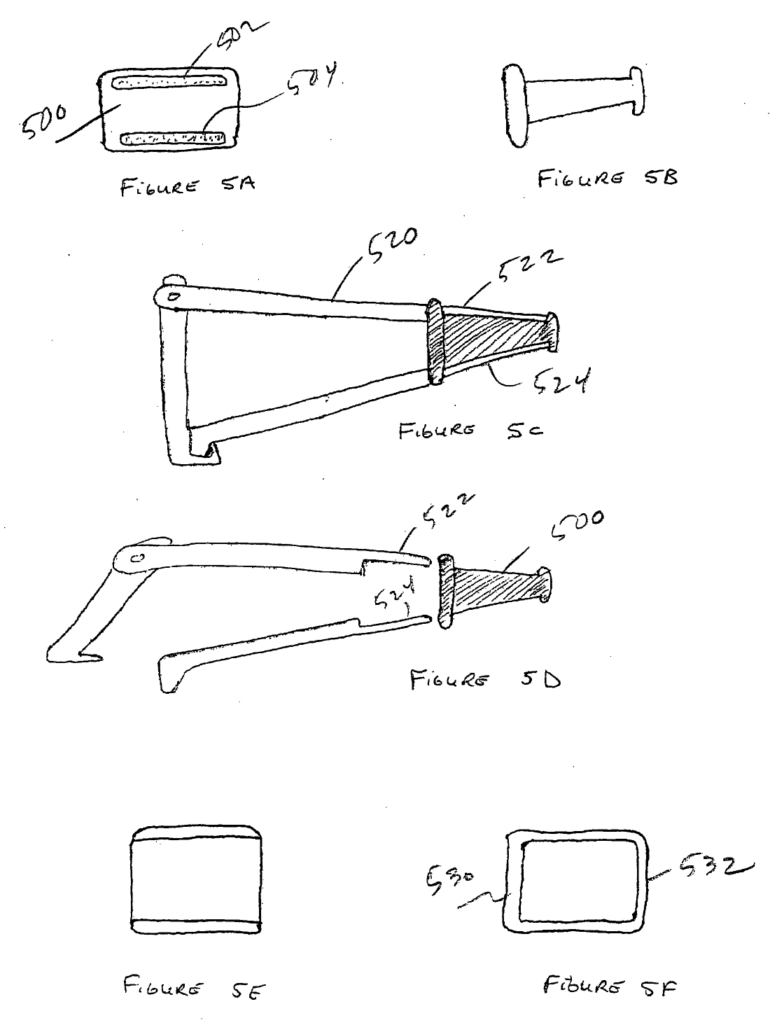

FIG. 4A is an anterior view of the spine, an exploded view of an alternative embodiment of an ADR incorporating novel spikes 402, 404 used to attach the ADR to the vertebrae. For example, the proximal end of the spikes could be threaded into threaded holes in the ADR. A flexible drill could be used to drill pilot holes for the modular spikes. FIG. 4B is a sagittal cross section of the spine and the embodiment of the invention drawn in FIG. 4A.

FIG. 4C is a coronal cross section of the spine and the embodiment of the invention drawn in FIG. 4A. The drawing illustrates a novel method of inserting the ADR 180 degrees to the method used in FIG. 4A. The novel method allows the insertion of spikes into the vertebra above or below the ADR. The lower ADR component 404 is held in the disc space by the upper ADR component. A convexity from one ADR component fits into a concavity in the other ADR component. The coupling between the ADR components prevents one ADR component from independently extruding from the disc space. FIG. 4D is a coronal cross section of the spine and an alternative embodiment of the invention drawn in FIG. 4C. The modular spikes diverge but may converge.

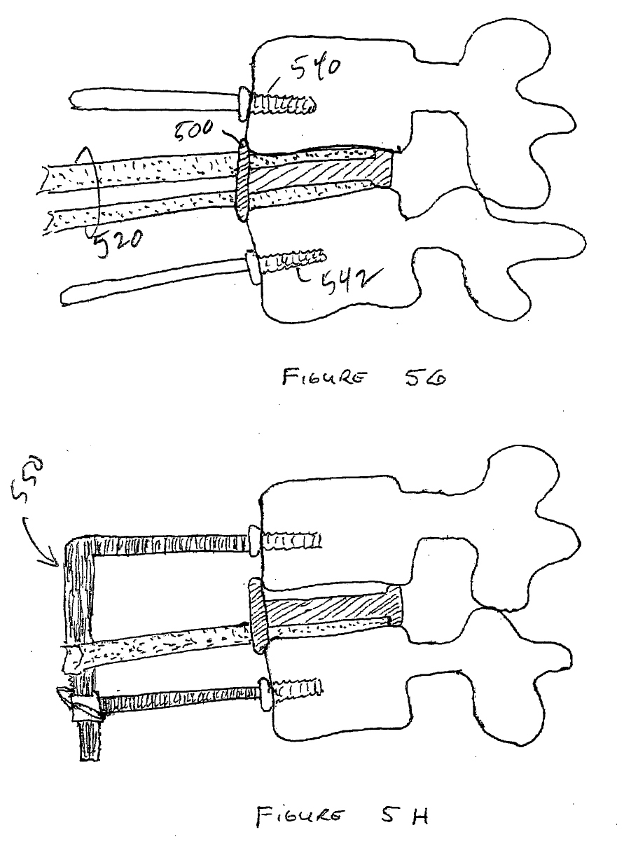

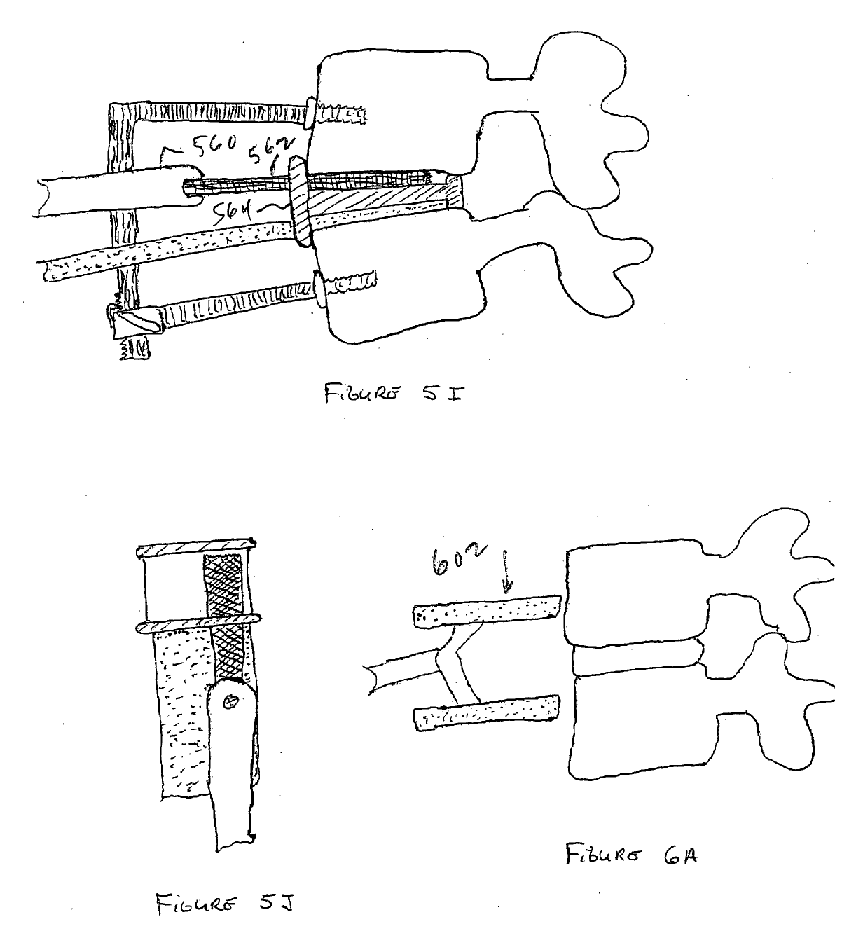

FIG. 5A is an anterior view of a novel cutting and distraction device 500 used to prepare the disc space for an ADR. The areas 502, 504 represent slots in the device. FIG. 5B is a lateral view of the embodiment of the device drawn in FIG. 5A. FIG. 5C is a lateral view of the device drawn in FIG. 5B and a tool 520 to hold the device. Projections 522, 524 from the holding tool fit into the slots of the cutting guide. FIG. 5D is an exploded lateral view of the embodiment of the invention drawn in FIG. 5C. FIG. 5E is a view of the top of the cutting guide drawn in FIG. 5A. FIG. 5F is a view of the top of an alternative cutting guide which has walls 530, 532 along the sides of the guide.

FIG. 5G is a sagittal cross section of the spine and a portion of the device drawn in FIG. 5C. The cutting guide 500 and holding tool 520 have been impacted into the disc space. The wedge shape of the assembled device separates the vertebrae. Prior-art distraction screws 540, 542 are shown in the anterior portion of the spine.

FIG. 5H is a sagittal cross section of the spine, a portion of the device drawn in FIG. 5G, and a prior-art distraction apparatus 550. The superior component of the holding tool has been removed. The prior art distraction apparatus is used to maintain distraction of the disc space after the superior component of the holding tool has been removed. The prior-art distraction device fits over the distraction screws drawn in FIG. 5G.

FIG. 5I is a sagittal cross section of the spine, a portion of the device drawn in FIG. 5G, a prior-art distraction device, a portion of a saw 560 and saw blade 562. The saw blade fits into the slot in the cutting guide. The projection 564 from the posterior portion of the cutting guide helps maintain distraction of the disc space and prevents the saw blade from entering the spinal canal. FIG. 5J is a superior view of the cutting guide, a portion of the holding tool, and the saw drawn in FIG. 5I.

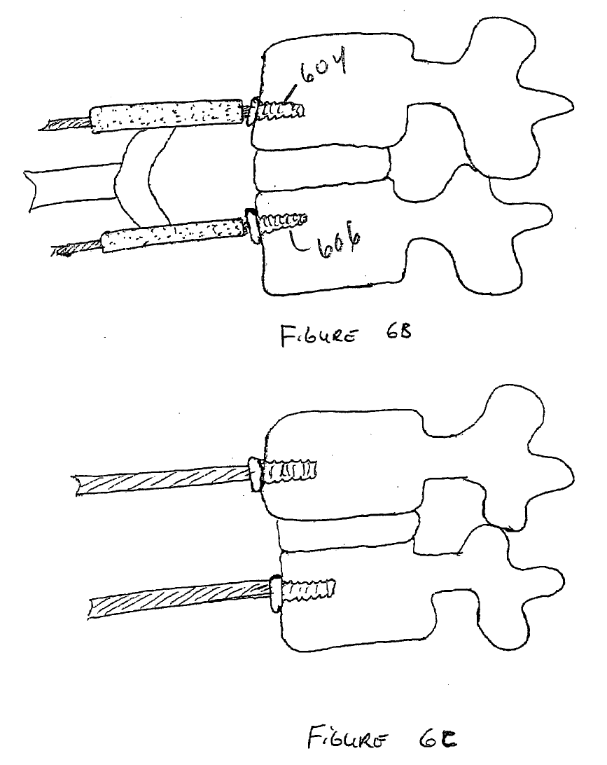

FIG. 6A is a lateral view of the spine and a novel guide 602 used to align the prior-art distraction screws drawn in FIG. 5G. The guide 602 assures the distraction screws are placed parallel to one another. FIG. 6B is a partial sagittal view of the spine and the novel guide drawn in FIG. 6A. Distraction screws 604, 606 can be seen coursing through the guide and into the spine. FIG. 6C is a partial sagittal view of the spine after insertion of the distraction screws.

FIG. 6D is a partial sagittal view of the spine, distraction screws, and a novel distraction apparatus 610. The sleeves of the distraction apparatus diverge as the sleeves approach the body of the apparatus. The novel shape of the distraction apparatus force the spine into a lordotic position as the sleeves are placed over the distraction screws.

FIG. 6E is partial sagittal view of the spine, the embodiment of the invention drawn in FIG. 6D, an ADR 620, and a novel ADR holder 622. The novel ADR holder has a projection 624 that cooperates with the anterior portion of the spine to assure proper placement of the ADR. For example, the projection could assure the ADR is recessed 2mm into the disc space. Alternatively, the projection could assure the front portion of the ADR is flush with the anterior surface of the spine. The projection also prevents inadvertent insertion of the ADR into the spinal canal.

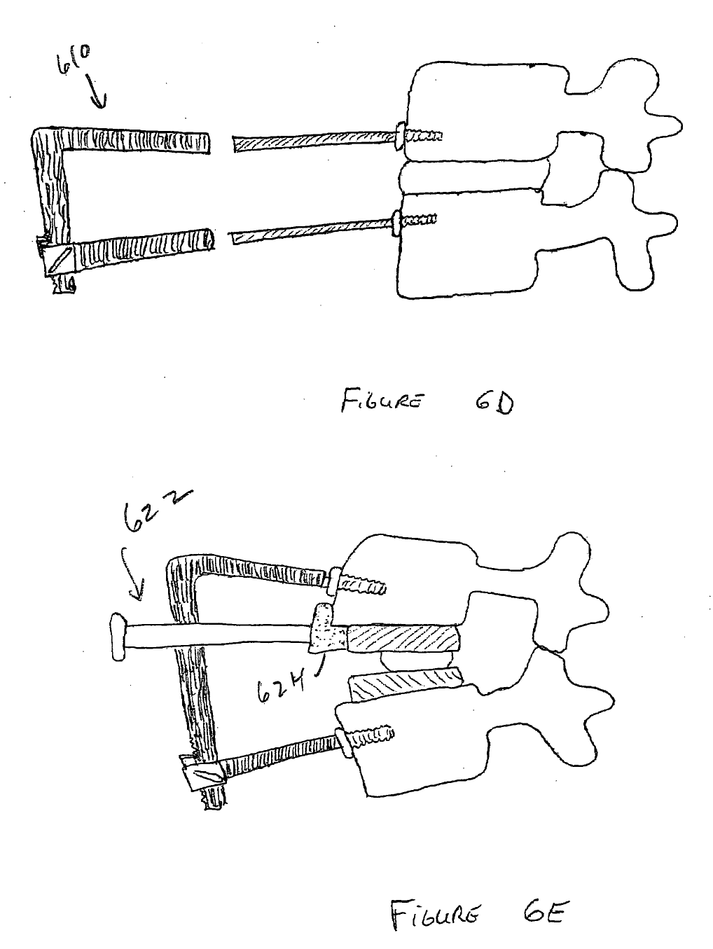

FIG. 7A is superior view of an ADR and an alternative embodiment of the invention providing pliers-like holder 702 used to grasp the side of an ADR 704. The novel holder may also have a component that cooperates with the anterior surface of the spine. FIG. 7B is a superior view of a novel “slap hammer” that may be used with the holder drawn in FIG. 7A to extract the ADR. The portion 710 slides along the shaft 712 of the device. FIG. 7C is a view of the assembled devices drawn in FIGS. 7A and 7B. The hook 720 of the slap hammer device fits into the axilla of the ADR holder. A nut 722 on the holder is advanced to hold the two tools together.

FIG. 7D is an exploded lateral view of an ADR 730 and a portion of the pliers-like device drawn in FIG. 7A. Arms from the holder attach to both ADR components. Projections from the holder fit into holes in the ADR. The drawing illustrates use of round and square holes 740, 742 and a projection 744. FIG. 7E is lateral view of an assembled ADR and holder. The holding tool holds the ADR components in a wedge shape. FIG. 7F is lateral view of the spine and the ADR and holder drawn in FIG. 7E. The ADR has been impacted into the disc space.

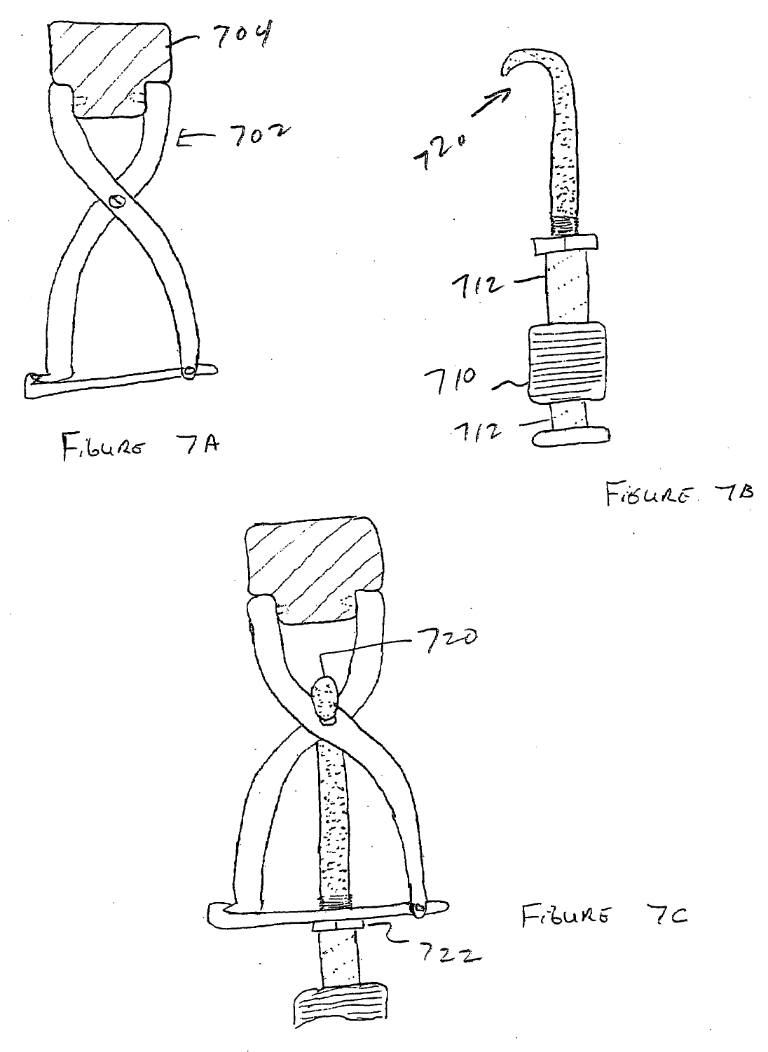

FIG. 8A is lateral view of the cutting guide drawn in FIG. 5F. FIG. 8B is a coronal cross section of the cutting guide drawn in FIG. 8A, a saw blade 802, and a portion of the holding tool 804 drawn in FIG. 5C. The walls along the side of the cutting guide prevent the saw blade from cutting too lateral in the disc space. The walls of the cutting guide also help maintain distraction of the disc space. FIG. 8C is lateral view of the spine, and the embodiment of the device drawn in FIG. 8B.

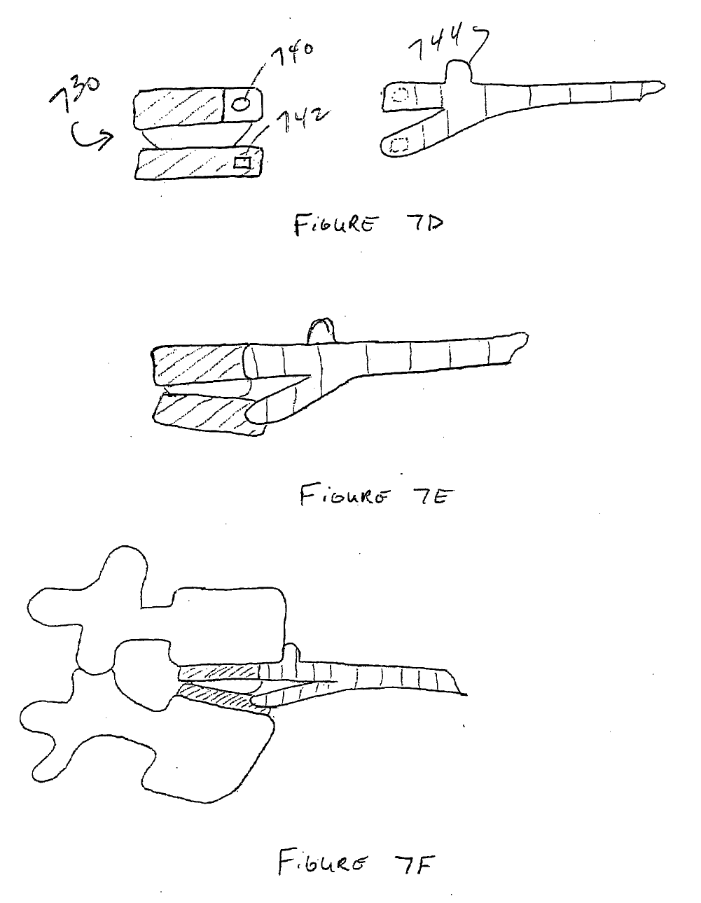

FIG. 9A is an anterior view of a novel component 902 used to prevent movement of ADRs. FIG. 9B is a lateral view of the device drawn in FIG. 9A. FIG. 9C is a lateral view of an ADR and the device drawn in FIG. 9B. The modular device 902 is attached to the front of an ADR. FIG. 9D is an exploded lateral view of the embodiment of the invention drawn in FIG. 9C.

FIG. 9E is a partial sagittal view of the spine, the embodiment of the invention drawn in FIG. 9C, and plates and screws. The novel invention is used to immobilize the spine after ADR insertion. For example, the device may be used in conjunction with other spinal fixation devices 990 to achieve a spinal fusion.

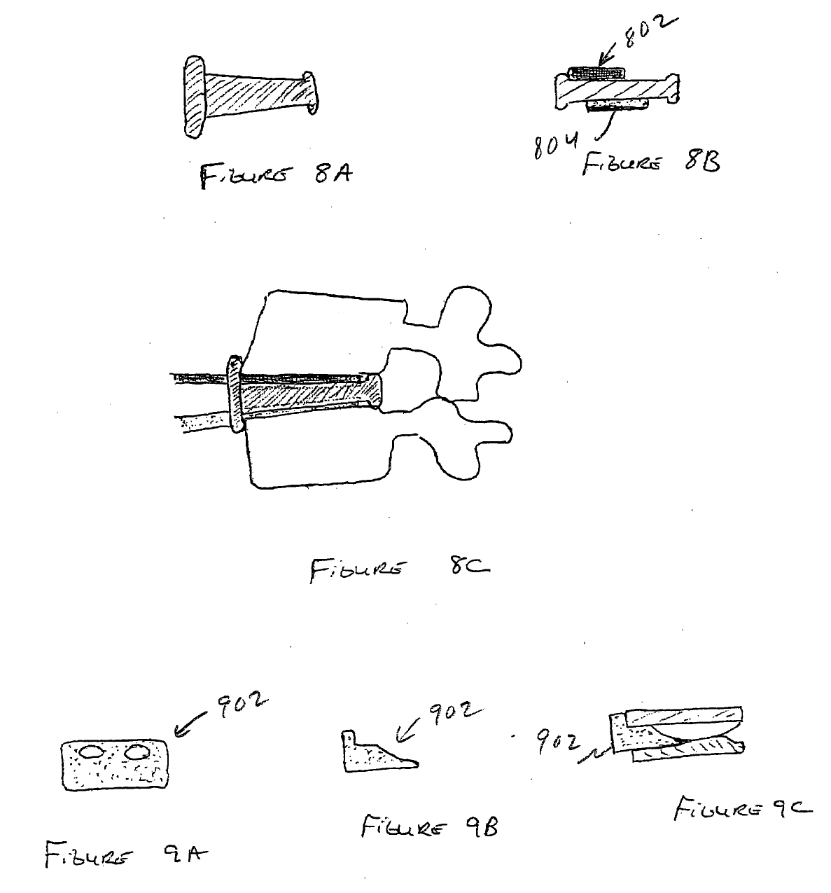

FIG. 10A is a lateral view of a novel ADR, wherein the posterior portion of the superior ADR component has been beveled or rounded at 1002 to better fit patients' anatomy. For example, the novel ADR shape fits the cervical disc space better than prior-art ADRs. FIG. 10B is a lateral view of an alternative embodiment wherein the posterior portion 1004 of the inferior ADR component has been beveled or rounded. The novel shape fits the lumbar disc space better than prior-art ADRs.

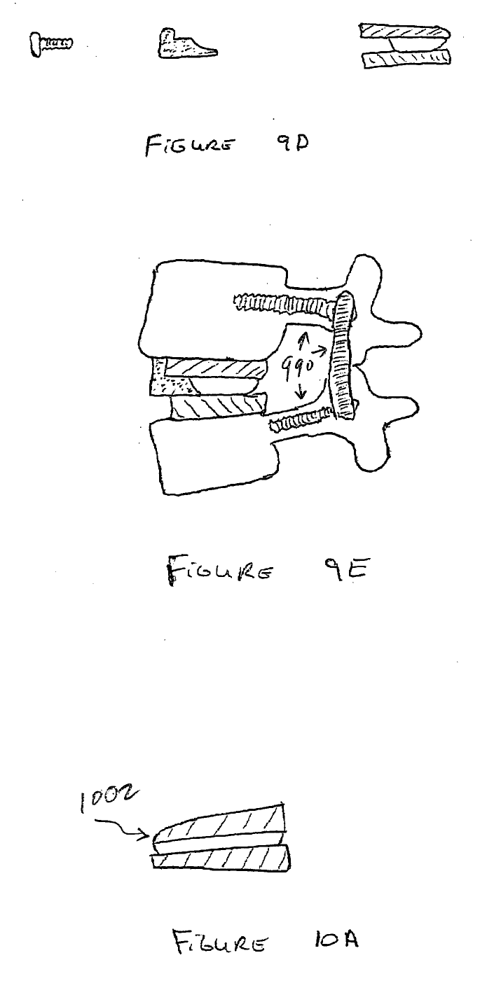

FIG. 11A is an anterior view of an alternative embodiment of the ADR drawn in FIG. 10B wherein both ADR components 1102, 1104 have holes that accept a single screw or spike. FIG. 11B is a lateral view of the ADR drawn in FIG. 11A. Screws 1106, 1108 have been inserted into the ADR. The screws are attached to the ADR. The screws preferably diverge to hold the ADR in the disc space. FIG. 11C is an anterior view of the spine showing how the screws or spikes are offset to avoid impingement between adjacent screws or adjacent ADR endplates.

FIG. 12 is a superior view of a pliers-like holder that fits into triangular or other shaped depressions in the sides of the ADR 1212. The posterior portion 1220 of the ADR may be tapered to facilitate insertion into the disc space.

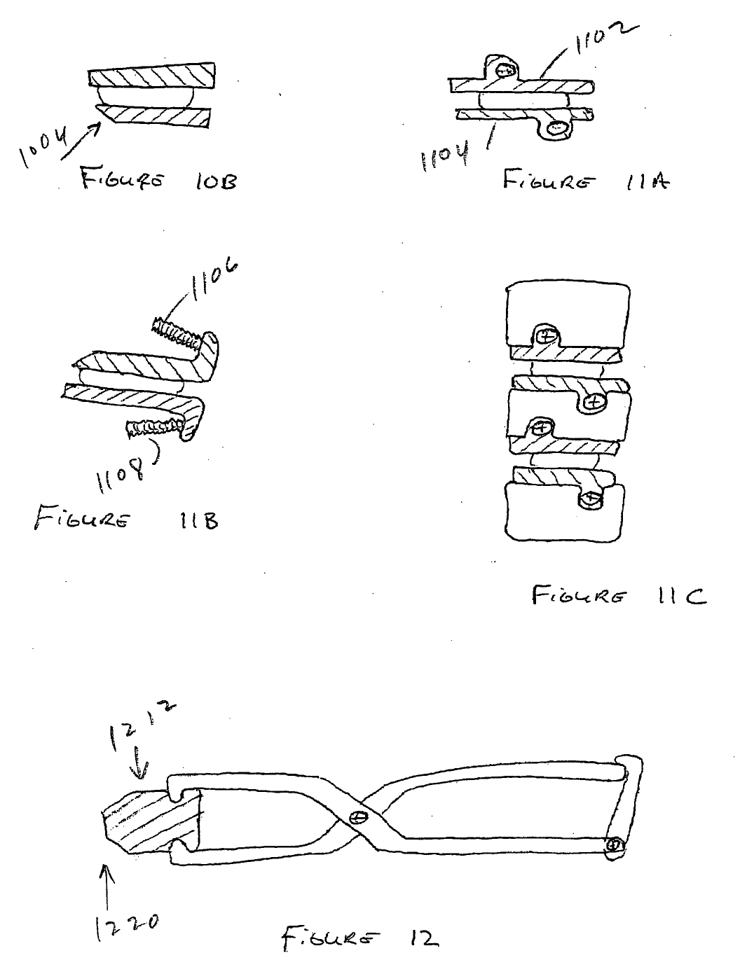

FIG. 13A is a lateral view of an alternative distractor/cutting guide according to the invention. FIG. 13B is a lateral view of the spine and the device. The handle 1302 of the guide has been folded inferiorly to allow insertion of a saw 1304. The saw blade 1306 fits through a slot in the anterior portion of the guide. FIG. 13C is a partial sagittal cross section of the spine and the device drawn in FIG. 13B. The saw is used to remove a portion of the vertebra superior to the ADR. Saw blades may be provided in various lengths in accordance with the level of the spine or other physical considerations. Note how the saw impinges against the front of the guide to limit the depth of saw blade insertion. FIG. 13D is a lateral view of the spine. The dotted line represents the portion of the superior vertebra that is cut from the superior vertebra.

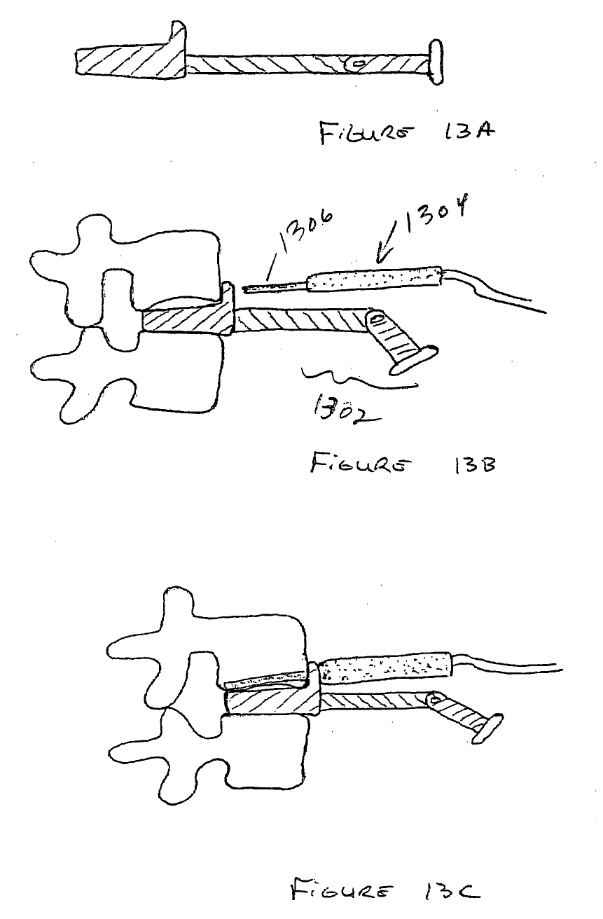

FIG. 14A is a lateral view of the spine and an alternative distractor/guide 1402 which is limited to the front portion of the disc space. FIG. 14B is a partial sagittal cross section of the spine and the device drawn in FIG. 14A. The drawing illustrates the use of a saw to remove a portion of the vertebra superior to the ADR. FIG. 14C is view of the top of a portion of the guide drawn in FIG. 14A. FIG. 14D is a view of the top of the guide drawn in FIG. 14C. A saw blade 1404 has been inserted through the slot in the front of the guide.

FIG. 15A is an anterior view of an alternative ADR including screws used to attach the ADR to the vertebra above 1502, 1504 and below 1506 the ADR. The screws are connected to the ADR by hinged plates as described in my co-pending application, U.S. Ser. No. 60/538,179. The invention teaches the use of a single screw to hold one of the ADR endplates. FIG. 15B is a lateral view of the ADR drawn in FIG. 15B.

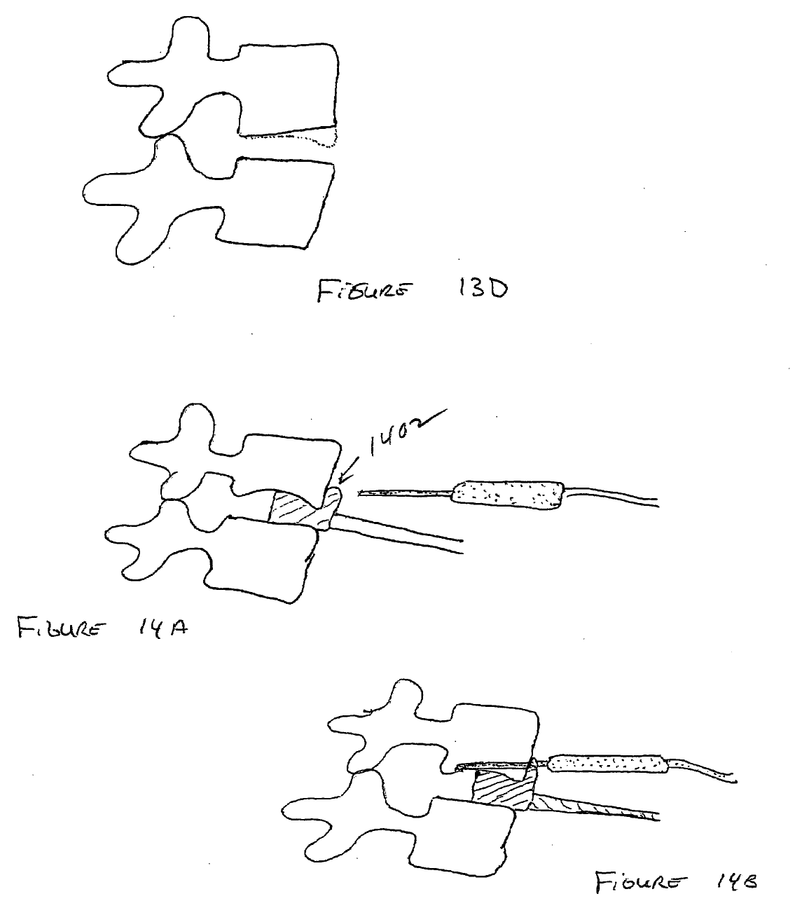

FIG. 16A is an anterior view of an alternative embodiment wherein a single screw is used in each ADR component. FIG. 16B is a lateral view of the spine and the embodiment of the ADR drawn in FIG. 16A. The screws 1602, 1604 pass through a angled portion of the ADR. The angled portion of the ADR courses between the vertical anterior surface of the ADR and the horizontal intradiscal portion of the ADR. FIG. 16C is a lateral view of the spine and an alternative embodiment which does not extend forward of the vertebral bodies 1610, 1612.

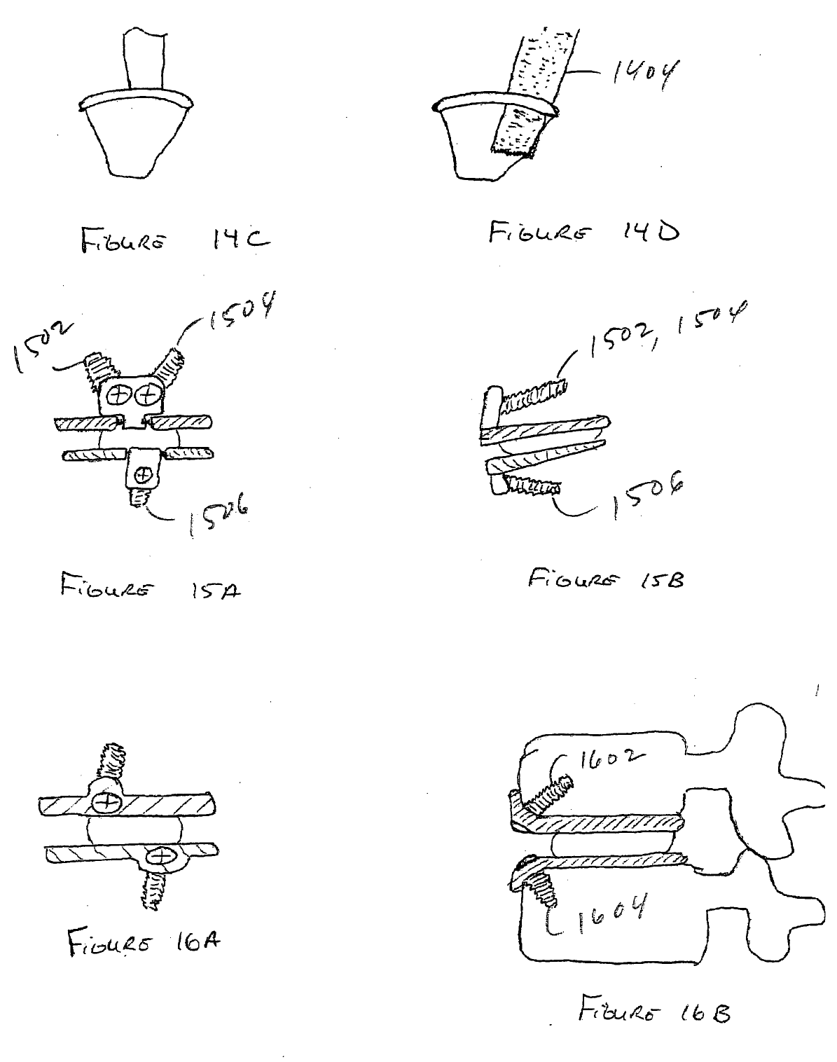

FIG. 17A is a view of the bottom of the upper ADR Endplate (ADR EP) drawn in FIG. 16A. The area 1702 represents a concave articulating surface in the upper plate. The area 1704 represents a hole the ADR. The hole cooperates with a tool that aligns the ADR in the disc space. FIG. 17B is a view of the top of the lower ADR EP drawn in FIG. 16B. The area 1710 represents a convex articulating surface in the lower plate. FIG. 17C is a coronal cross section of the ADR drawn in FIG. 16A. The alignment holes 1720, 1722 are directly opposite of one another.

FIG. 17D is a lateral view of an instrument 1730 used to align the ADR EPs drawn in FIG. 17C. FIG. 17E is a coronal cross section of the ADR drawn in FIG. 17C and the alignment tool drawn in FIG. 17D. The tool cooperates with the holes in the ADR EPs to align the ADR EPs.

FIG. 18 is an anterior view of an alternative embodiment wherein the anterior surfaces of the ADR EPs have grooves or surface markings to align the ADR EPs. The tool drawn in FIG. 17D may fit into the grooves on the anterior surfaces of the ADR EPs.

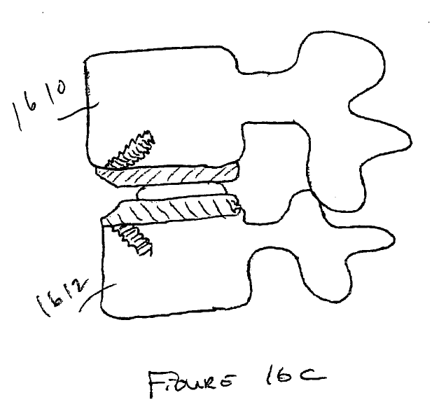

FIG. 19A is an exploded lateral view of an ADR and alternative embodiment of the invention related to that drawn in FIG. 9D. The device 1900 is attached to the upper and the lower ADR endplates (EPs 1902, 1904). Screws 1910 may be used to attach the device to the ADR EPs. Alternative methods may be used to fasten the device to the ADR EPs. For example, the fastening method could include the use of shape memory materials such as Nitinol.

FIG. 19B is a lateral view of the embodiment of the invention drawn in FIG. 19A and an ADR. The component 1900 prevents the ADR EPs from moving towards one another. For example, if the component was placed on the posterior portion of an ADR it would prevent spinal extension through the ADR. The screws and the component cooperate to prevent other motions such as lateral bending and axial rotation. The screws cooperate with the component to prevent the ADR EPs from separating. Thus, posterior placement of the device also prevents spinal flexion through the device. The device is preferably made of a biocompatible metal such as titanium or chrome cobalt. The device can also made of other biocompatible materials. FIG. 19C is a posterior view of the device and an ADR.

FIG. 20 is the view of the top of an ADR and embodiments of the invention drawn in FIG. 19A, showing how devices may be placed on the anterior 2002, posterior 2004, lateral 2006, and/or posterior-lateral portions 2008 of an ADR.

FIG. 21 is a posterior view of a an ADR having holes 2102, 2104 to receive the embodiment of the invention drawn in FIG. 19A. The ADR also has novel projections 2110, 2112 from the ADR EPs. The projections are placed directly over the holes in the ADR EPs. Surgeons could use fluoroscopy to identify the projections from the ADR EPs. The location of the projections directs surgeons to the holes in the ADR EPs. Surgeons could use the alignment apparatus to minimize the length of the incision required to insert the device.

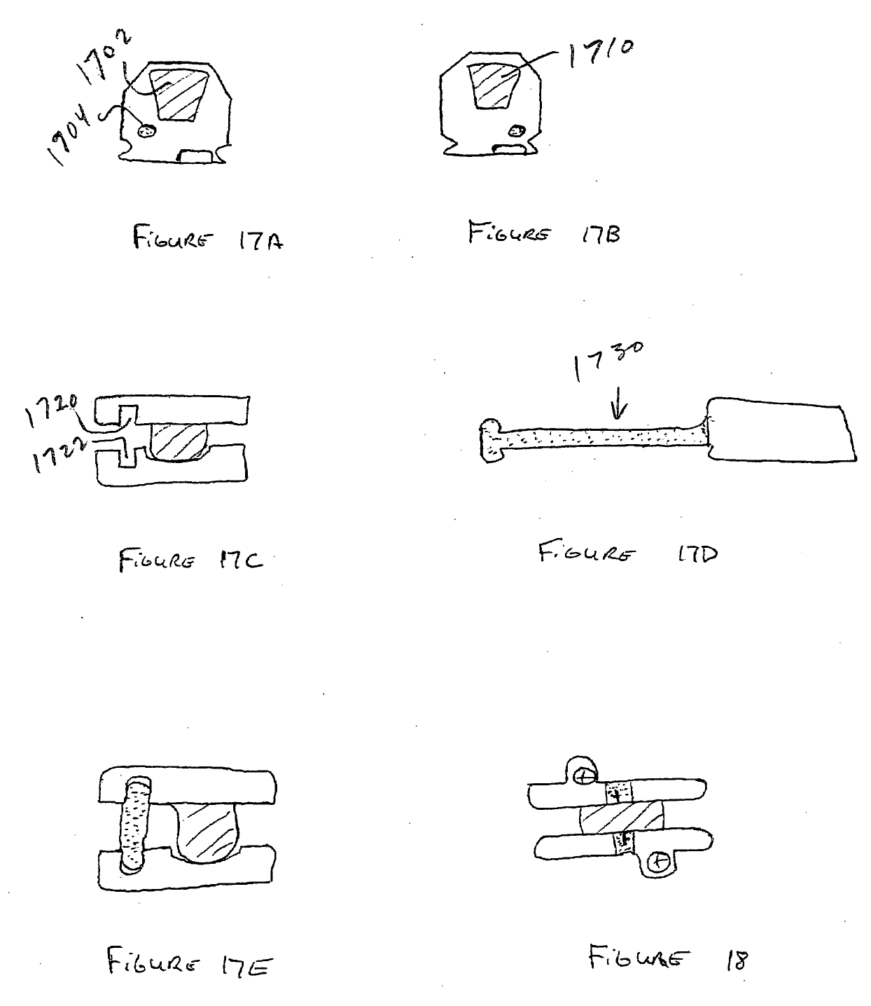

FIG. 22 is a lateral view of an alternative embodiment of the invention and a sagittal cross section through a novel ADR 2204. The device 2202 has elastic properties. The device is impacted over the ADR EPs to arrest movement through the ADR. Like the embodiment of the invention drawn in FIG. 19A, the device fastens to the ADR EPs to prevent flexion, extension, lateral bending, translation, and/or axial rotation through the ADR.

FIG. 23A is an exploded lateral view of an alternative embodiment of the invention wherein threaded hook component 2302 passes through a blocker component 2304 and a hook component 2306. FIG. 23B is a lateral view of the embodiment of the invention drawn in FIG. 23A and an ADR. The assembled device is tightened to clamp onto the upper and lower ADR EPs. The hook portions of the device may fit into recesses or holes in the ADR EPs. The device is tightened over the ADR by advancing the nut along the threaded portion of the superior hook component.

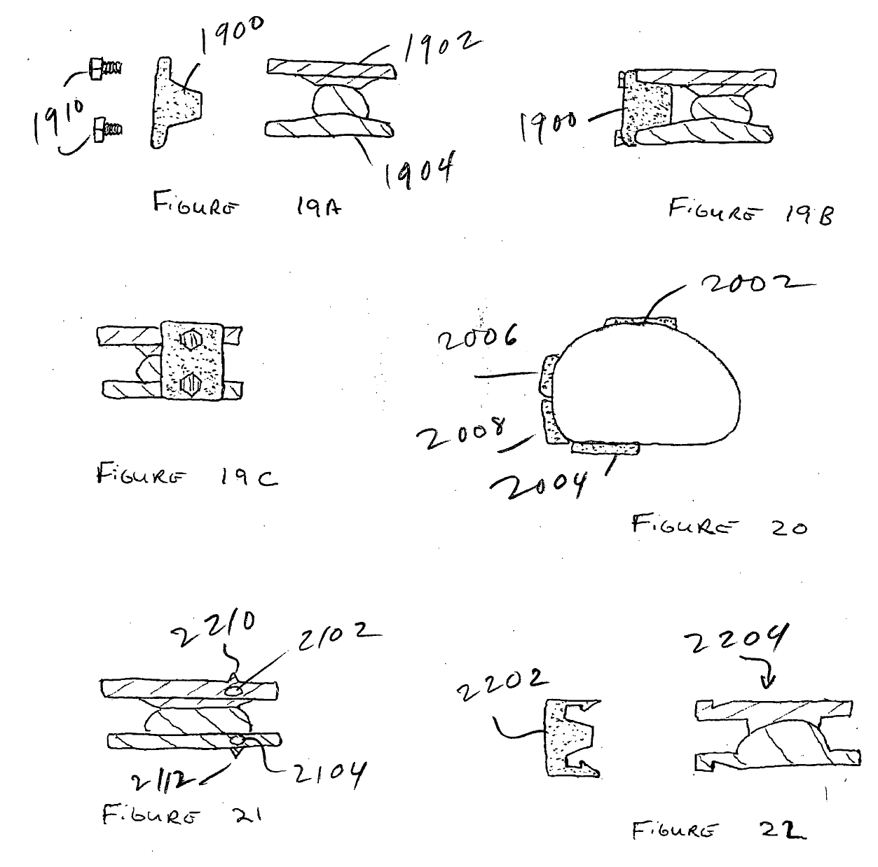

FIG. 24A is an exploded lateral view of an alternative embodiment of the invention drawn in FIG. 23A. FIG. 24B is a lateral view of the embodiment of the invention drawn in FIG. 24A and a novel ADR. The device 2402 is fastened to the ADR EPs with screws 2410. The stiffness of the device and the rigid attachment to the ADR EPs prevents movement through the ADR.

FIG. 24C is a view of the top of the embodiment of the ADR drawn in FIG. 24B. The rectangular opening 2402 represents a space in the ADR EP that is filled with a bone growth promoting substance. BMP soaked collagen sponges could be used to promote fusion across the ADR. Rectangle 2404 with the dots represents the embodiment of the device drawn in FIG. 24A. The two rectangles 2406, 2408 represent novel removable plugs in the ADR EPs. The plugs prevent bone from growing into the openings in the ADR EPs. The plugs are removed during revision surgery to place the embodiment of the device drawn in FIG. 24A and to insert bone growth promoting substances. FIG. 24D is a posterior view of the embodiment of the invention drawn in FIG. 24C.

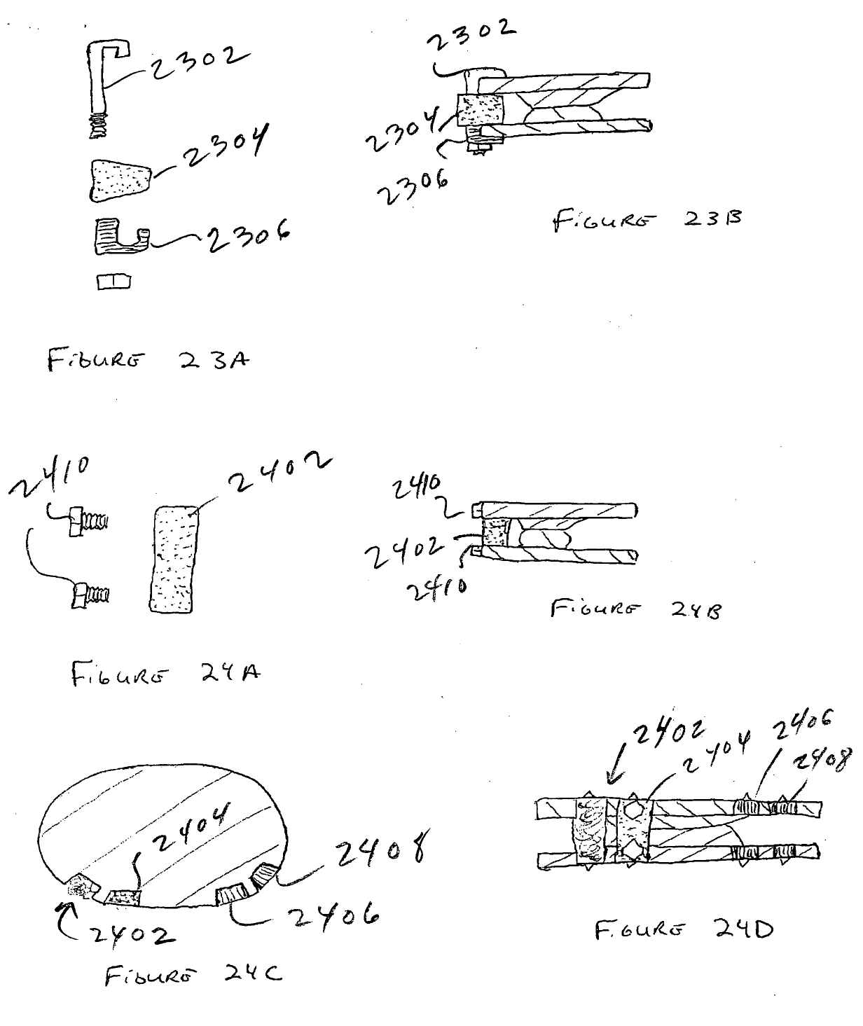

FIG. 25A is an exploded lateral view of an alternative embodiment of the invention, and FIG. 25B is a sagittal cross section of the embodiment of the invention drawn in FIG. 25A and an ADR. Screws 2502, 2504 are threaded through the blocker component 2510 represented by the dotted area of the drawing and into threaded holes in the ADR EPs. Alternatively, shape memory technology could be used to attach the device to the ADR EPs.

FIG. 26A is an exploded lateral view of an alternative embodiment of the invention, and FIG. 26B is a lateral view of the embodiment of the invention drawn in FIG. 26A and a sagittal cross section through an ADR. The hook portions 2602 of the device 2604 pass through holes or slots in the ADR EPs 2610, 2612. The hook portions of the device attach the device to the ADR and hold a blocker component between the ADR EPs. The inferior hook component slides along the shaft extending from the superior hook component. A set-screw passes through the lower hook component and against the shaft of the upper hook component. The screw is tightened to clamp the device 2620 to the ADR.

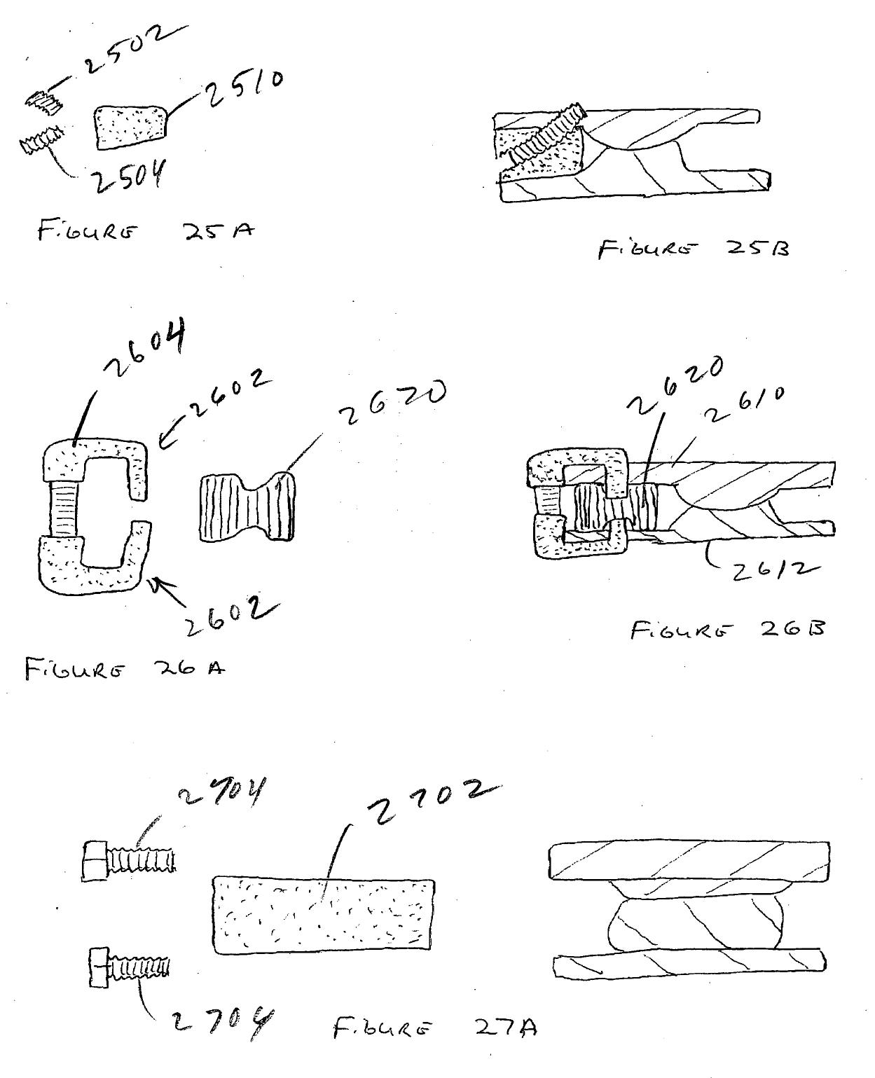

FIG. 27A is an exploded lateral view of an alternative embodiment of the invention and an ADR. FIG. 27B is a lateral view of the embodiment of the invention drawn in FIG. 27A. The rectangular shaped component 2702 fits into slots in the ADR EPs. The screws 2704 prevent the rectangular shaped component from backing out of the ADR.

FIG. 27C is a posterior view of the embodiment of the invention drawn in FIG. 27B. The screws have not been inserted into the ADR EPs. FIG. 27D is a partial posterior view of an alternative embodiment of the invention related to that drawn in FIG. 27C. The superior and inferior surfaces of the rectangular shaped component have projections 2730, 2732 that cooperate with the slots in the ADR EPs to help lock the component in the ADR EPs.

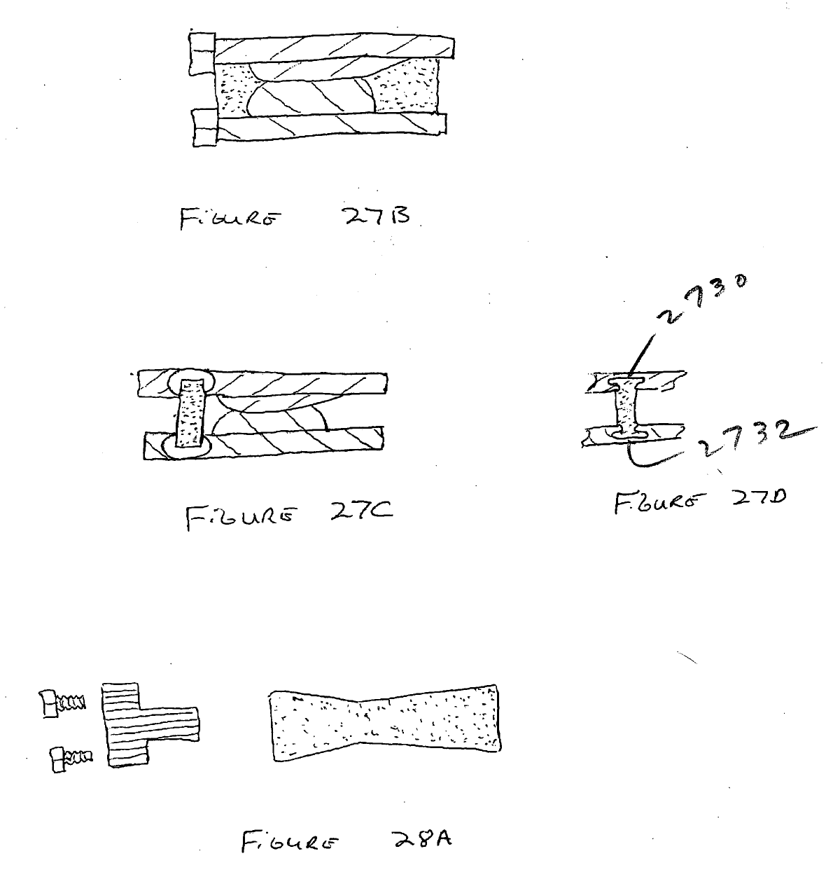

FIG. 28A is an exploded lateral view of an alternative embodiment of the invention, and FIG. 28B is an exploded view of the top of the embodiment of the invention drawn in FIG. 28A. FIG. 28C is a lateral view of the embodiment of the invention drawn in FIG. 28B and an ADR. The wedge component 2802 is drawn in its first position. FIG. 28D is a lateral view of the embodiment of the invention drawn in FIG. 28C. The wedge component has been rotated 90 degrees. The wedge component preferably fits into slots in the ADR EPs. The walls of the slots may be configured to facilitate rotation if the wedge component in one direction.

FIG. 28E is a view of the top of an ADR EP and the embodiment of the invention drawn in FIG. 28B. A forked-shaped component 2810 is attached to the ADR EP. The arms of the forked shaped component straddle the sides of the wedge component to prevent counter rotation of the wedge component. FIG. 28F is a lateral view of the embodiment of the invention drawn in FIG. 28E, wherein the forked shaped component has been fastened to the ADR EPs. The wedge component may course from anterior to posterior or obliquely across the ADR EPs in alternative embodiments of the invention.

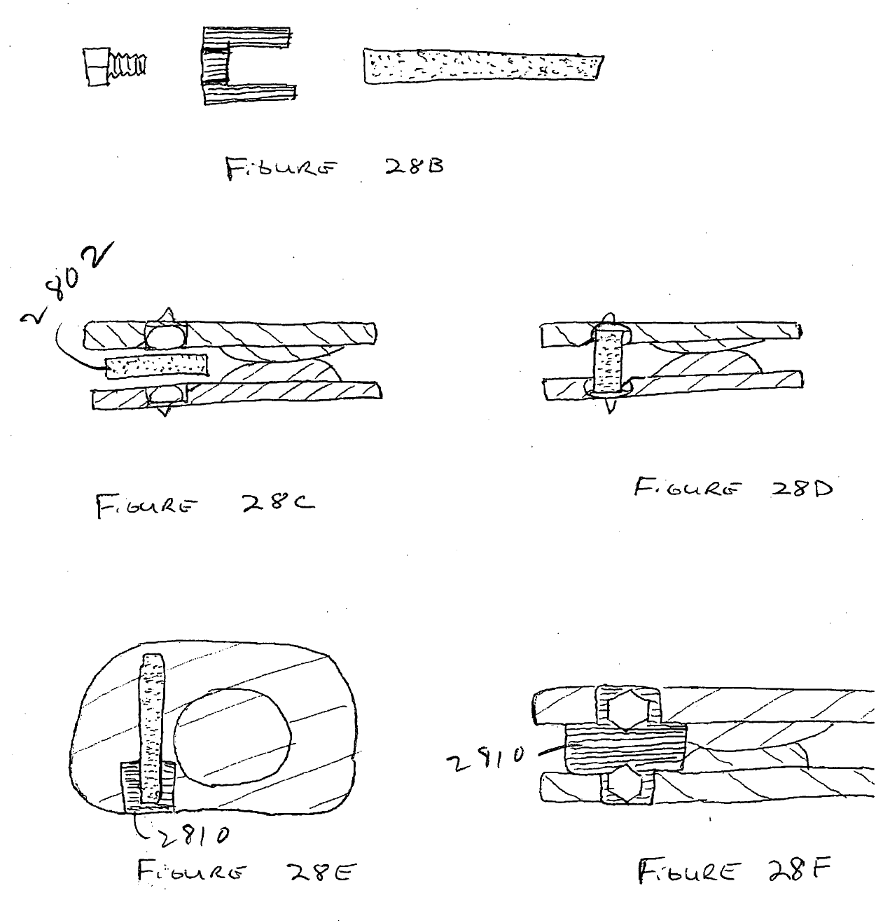

FIG. 29A is a lateral view of an alternative embodiment of the invention wherein a wedge-blocker component 2902 expands after placement of the device between the ADR EPs. Expansion of the device in-situ eliminates the need to rotate the device in-situ. FIG. 29B is a lateral view of the embodiment of the invention drawn in FIG. 29A. A threaded wedge component 2904 has been advanced to expand the tip of the blocker component. The device is placed between ADR EPs to prohibit movement across the ADR. FIG. 29C is a sagittal cross section of the embodiment of the invention drawn in FIG. 29B.

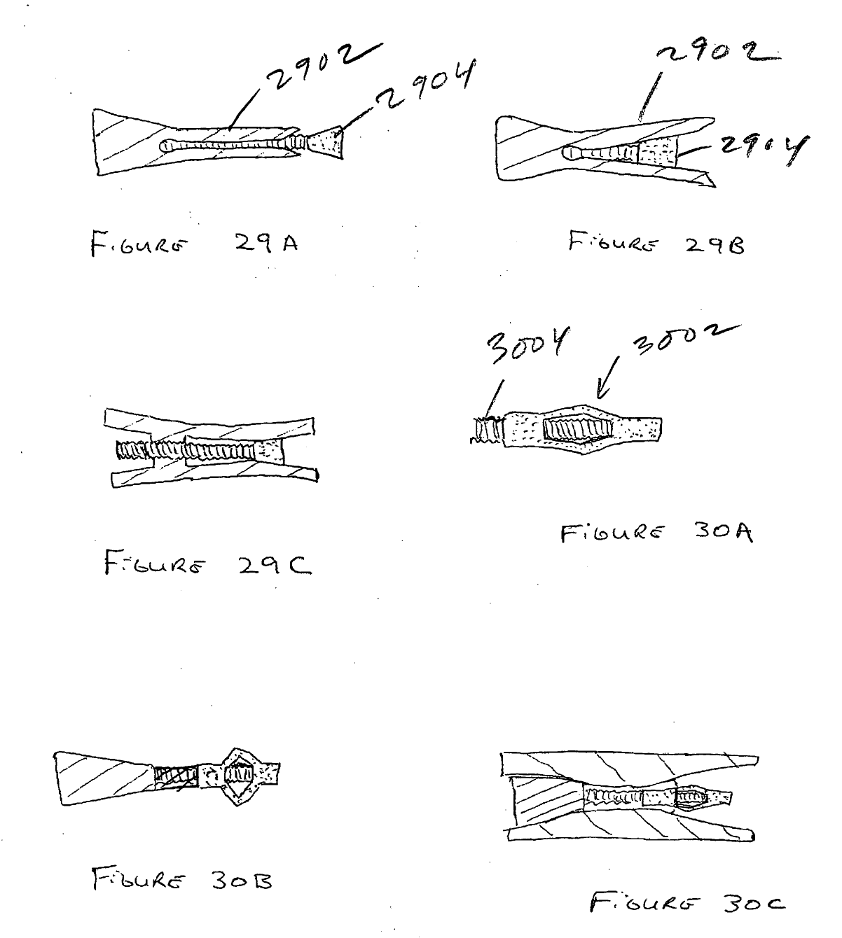

FIG. 30A is a lateral view of the tip of an alternative embodiment of the invention, and FIG. 30B is a lateral view of the embodiment of the invention drawn in FIG. 30A. The tip of the device 3002 expands as the screw 3004 is rotated. FIG. 30C is a lateral view of the embodiment of the invention drawn in FIG. 30B and an ADR. The device has been drawn in its contracted shape. The contracted shape facilitates insertion of the device.

FIG. 31A is a lateral view of an alternative embodiment of the device drawn in FIG. 25A. A screw 3102 is advanced from a hole in one ADR EP. The screw impinges against the second ADR EP 3104 to prevent motion across the ADR. FIG. 31B is a view of the top of the inferior ADR EP drawn in FIG. 31A. The ADR EP has two holes. FIG. 31C is a view of the top of the inferior ADR EP drawn in FIG. 31B. Screws have been advanced partially through the holes in the ADR EPs. One or more screws may be advanced across the superior and or inferior ADR EPs. The screws may impinge against the second ADR EP. Alternatively, the screws may be threaded into holes in the second ADR EP.

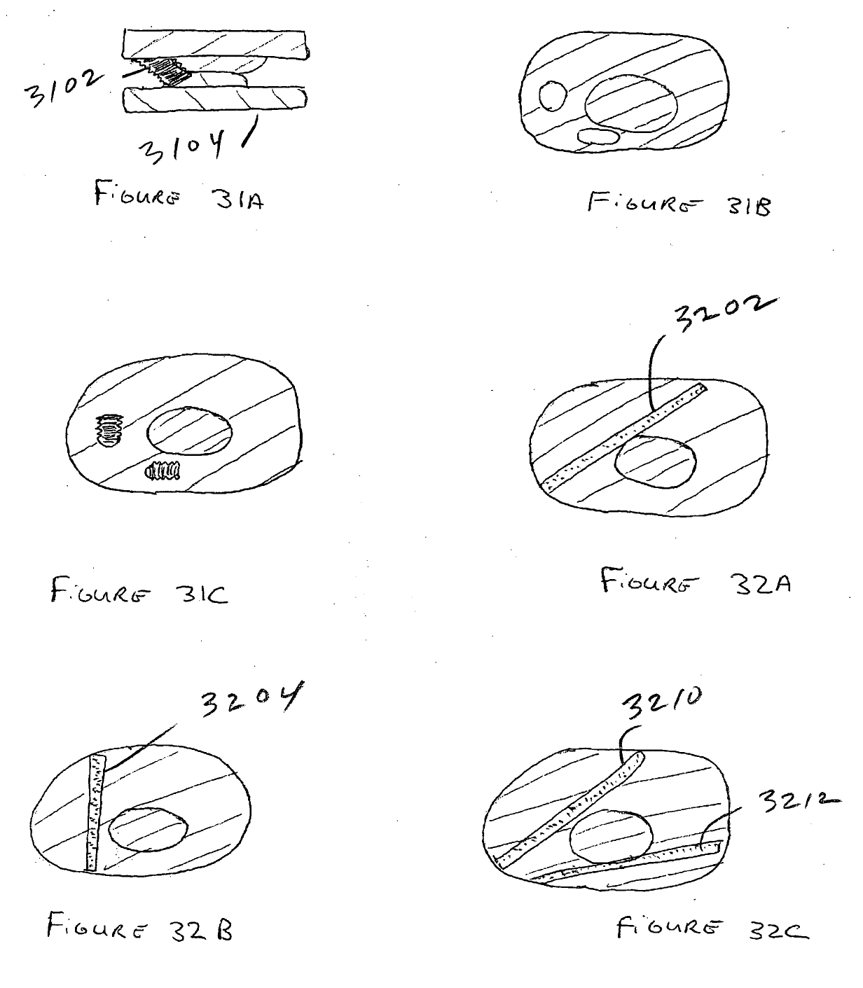

FIG. 32A is a view of the articulating side of the ADR EP drawn in FIG. 27A. The wedge component 3202 courses obliquely across the ADR. FIG. 32B is a view of the articulating side of an alternative embodiment of the invention drawn in FIG. 32A. The wedge component 3204 courses from anterior to posterior. The wedge component may be limited to one half of the ADR. Alternatively, the wedge component may be limited to one quarter of the ADR. For example, the wedge component may be limited to the posterior half of the left side of the ADR.

FIG. 32C is a view of the articulating side of an alternative embodiment of the invention wherein multiple wedge components 3210, 3212 course across the ADR. FIG. 32D is a view of the articulating side of an alternative embodiment of the invention drawn in FIG. 32C. The wedge component 3220 passes into the articulating surface of the ADR. A portion of the articulating surface may be removed to allow insertion of the wedge component. For example, a portion of a polyethylene component may be removed to insert a device according to the invention.

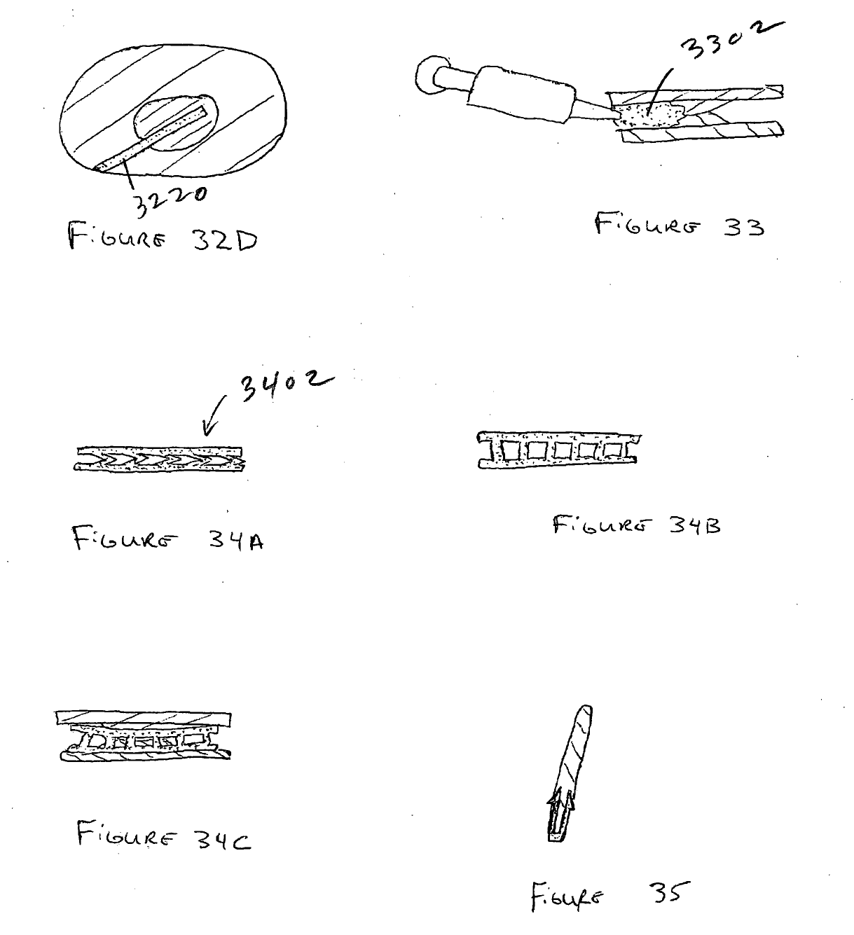

FIG. 33 is a lateral view of an ADR and an alternative embodiment of the invention wherein an in-situ curing polymer 3302 is injected between the ADR EPs. For example, polymethylmethacrylate (PMMA) may be injected between the ADR EPs. Dye may be injected into the space between the ADR EPs prior to injecting the PMMA. Injection of a radio-opaque dye helps surgeons determine where the PMMA will flow. Alternatively, in-situ curing polyurethane or other polymer may be injected between the ADR EPs. The cured polymer is preferably stiff.

FIG. 34A is a lateral view of an alternative embodiment of the invention wherein a frame-like device 3402 is made of a shape memory material. For example the device could be made of Nitinol. The device expands in-situ as the temperature of the body heats the device. FIG. 34B is a lateral view of the embodiment of the invention drawn in FIG. 34A. The device has expanded.

FIG. 34C is a lateral view of an ADR and the embodiment of the invention drawn in FIG. 34B. The device is drawn in its expanded shape. The device expands after it is placed between the ADR EPs. The device may be used with other embodiments of the invention. For example, the in-situ curing PMMA drawn in FIG. 33 could be injected into the ADR after inserted the shape memory frame.

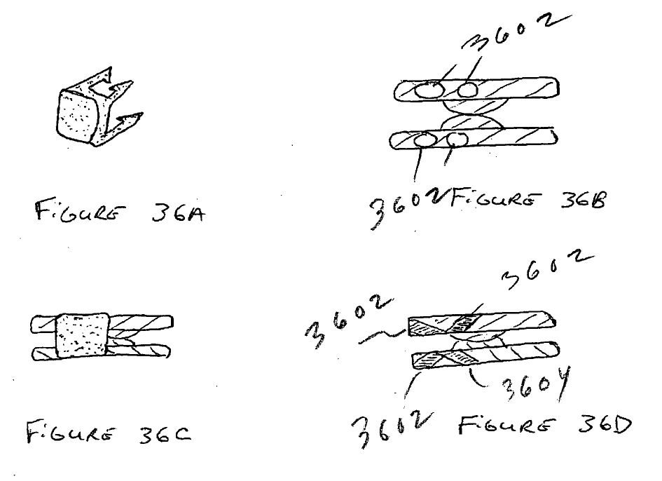

FIG. 35 is the view of the top of an alternative embodiment of the invention drawn in FIG. 27A. The rectangular component has an elastic spring clip projection. The clip holds the component in the ADR EP. The clip eliminates the need for screws to hold the component in the ADR. Such spring-loaded mechanisms could alternatively be incorporated into the ADR EPs. Elastic locking mechanisms could be incorporated into other embodiments of the invention.

FIG. 36A is an oblique view of an alternative embodiment of the invention drawn in FIG. 19A. FIG. 36B is a view of the posterior aspect of an alternative embodiment of the ADR drawn in FIG. 5A of my co-pending application U.S. patent application Ser. No. 10/741,290, the entire content of which is incorporated herein by reference.

FIG. 36C is a view of the posterior aspect of the embodiment of the ADR drawn in FIG. 36B and the posterior aspect of the embodiment of the invention drawn in FIG. 36A. The arms of the device drawn in FIG. 36A fit into holes 3602 of the ADR. The elastic properties of the device cooperate with the holes in the ADR to lock the device in the ADR. The device prevents movement through the ADR.

FIG. 36D is a sagittal cross section through the embodiment of the ADR drawn in FIG. 36B. The holes 3602 receive the arms of the device drawn in FIG. 36A. The holes also allow bone to grow across the ADR. Bone growth material may be placed between the holes. For example, a BMP soaked collagen sponge could be placed between the holes in the ADR EPs. A drill may be passed diagonally from a hole in one ADR to a hole in the second ADR EP. The drill causes the vertebrae to bleed. The blood from the vertebrae and the bone growth material stimulate spinal fusion across the ADR. The device drawn in FIG. 36A immobilizes the ADR EPs. Immobilization of the ADR EPs facilitates fusion across the ADR EPs. The holes could be filled with Polyethylene. The polyethylene prevents bone from growing across the ADR EPs. The polyethylene may be removed with a drill during revision surgery. BMP soaked sponges may be packed into the freshly drilled holes in the ADR EPs and the holes drilled into the vertebrae.

I claim:

1. A method of spinal arthroplasty, comprising the steps of:
installing an anchoring unit into a portion of a vertebrae during a first procedure; and
coupling an arthroplasty device to the anchoring unit as part of a subsequent procedure.

2. The method of claim 1, wherein the time between the first and subsequent procedures is sufficient to promote ingrowth in the anchoring unit.

3. The method of claim 1, wherein at least the first procedure is minimally invasive.

4. The method of claim 1, wherein the arthroplasty device is plate, rod or other component used for spinal fixation.

5. Arthroplasty apparatus, comprising:
an implant; and
a staple that holds the implant in position relative to a bone, the staple including prongs that converge or diverge upon entry.

6. The apparatus of claim 5, wherein:
the prongs extend through apertures in the implant: and
the apertures cause the prongs to converge or diverge upon entry.

7. The apparatus of claim 5, wherein the prongs are composed of a shape-memory material.

8. Arthroplasty apparatus, comprising:
an artificial disc replacement including a pair of opposing endplates; and
a device for immobilizing the endplates once in position.
